(19) United States
(12) Patent Application Publication
(10) Pub. No.: US 2019/0275184 A1
(43) Pub. Date: Sep. 12, 2019

(54) Recovery and recycle of Ethylene Oxide from Sterilization/Fumigation Processes

(71) Applicants: Joseph D. Duff, Louisville, KY (US); Joseph E. Paganessi, Burr Ridge, IL (US)

(72) Inventors: Joseph D. Duff, Louisville, KY (US); Joseph E. Paganessi, Burr Ridge, IL (US)

(21) Appl. No.: 15/821,376

(22) Filed: Nov. 22, 2017

(51) Int. Cl. A61L 2/20; C07D 301/32

(52) U.S. Cl. CPC A61L 2/206; C07D 301/32

(57) ABSTRACT

In the commercial processes of using ethylene oxides for sterilization and fumigation over 99% of the ethylene oxide used is the process goes unreacted and is discharged from the sterilization/fumigation chamber. This process allows for the capture of ethylene oxide by using temperature, pressure, and both temperature and pressure to condense, separate and recycle the liquefied ethylene oxide from the non-condensable gases. The invention allows the user to recover then recycle the bulk of the exhausted ethylene oxide significantly increasing its utilization thus reducing the quantity of ethylene oxide required to be on-site. As secondary benefits, both feed stock and mitigation costs are significantly reduced.

### CROSS REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The present invention relates to the recovery and recycling of ethylene oxide that is used in the sterilization of equipment.

### SUMMARY OF THE INVENTION

In the commercial processes of using ethylene oxides for sterilization and fumigation over 99% of the ethylene oxide used is the process goes unreacted and is discharged from the sterilization/fumigation chamber. This process allows for the capture of ethylene oxide by using temperature, pressure, and both temperature and pressure to condense, separate and recycle the liquefied ethylene oxide from the non-condensable gases. The invention allows the user to recover then recycle the bulk of the exhausted ethylene oxide which significantly increasing its utilization thus reducing the quantity of ethylene oxide required to be on-site. As secondary benefits, both feed stock and mitigation costs are significantly reduced. The range of molecular concentrations of the feed stock that where investigated is presented in Table 1. FIG. 1 is the potential ethylene oxide recovery versus system pressure and temperature for Feed Stock A which is given in Table 2.

### BRIEF DESCRIPTION OF THE DRAWINGS

FIG. 1 is a chart showing Ethylene Oxide Recovery versus condensing temperature and operating pressure for Feed A.

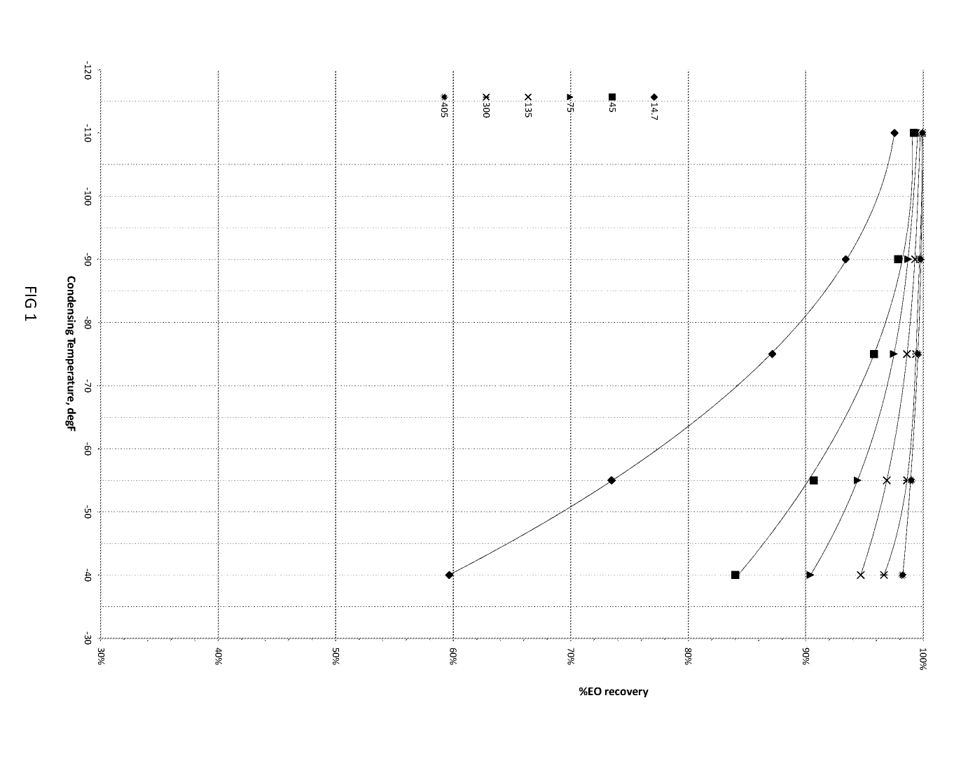

FIG. 2 is a flow chart of the generalized ethylene oxide recovery system process.

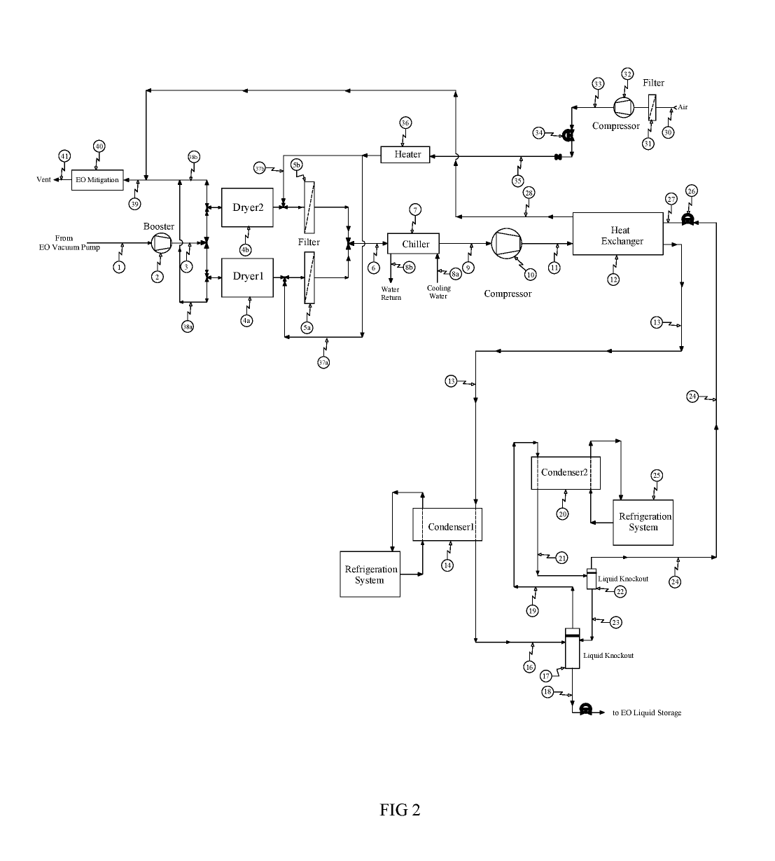

### DETAILED DESCRIPTION OF THE INVENTION

The feed stream (1) originates at the discharge of the sterilization/fumigation chamber vacuum pump. At this point, the gas stream containing unreacted ethylene oxide is between 120•150 degrees F., slightly above atmospheric pressure, and has a composition range present in Table 1. A booster pump (2) is used to increase the gas stream pressure to between 20 and 30 psia. The gas temperature increases by about 50 degrees. Using a series of three-way diverter valves, the discharged gas stream (3) is directed to one of the two parallel molecular sieve dryers (4a or 4b) where moisture is removed to give a dew point of less than or equal to −80 degrees, followed by a filter to remove any particulate matter. The dehydrated, filtered gas stream (6) is fed into a countercurrent heat exchanger (7) where the temperature of the gas stream (9) is reduced to 65 to 85 degrees using process cooling water (Sa) which is 60 to 80 degrees. Gas stream (9) is then directed to the inlet of a compressor (10) where its pressure is increased to the desired operating pressure based on the required condensing temperature for the ethylene oxide to achieve the desired recovery efficiency.

TABLE 1
Molecular Species Concentrations Ranges for Process Feed Gases

| | Concentration Range | |
|---|---|---|
| Component | Lower | Upper |
| Nitrogen | 4.6M % | 87.1M % |
| Oxygen | 0.22M % | 20.8M % |
| Carbon Dioxide | 0M % | 0.04M % |
| Argon | 0.01M % | 0.92M % |
| Water | 0.10M % | 11.9M % |
| Ethylene Oxide | 0.70M % | 82.2M % |

TABLE 2
Molecular Species Concentration for Feed A

| | Stream, M % | | |
|---|---|---|---|
| Component | 1 | 2 | 3 |
| Nitrogen | 59.99% | 87.14% | 81.34% |
| Oxygen | 0.70% | 0.22% | 13.49% |
| Carbon Dioxide | 0.00% | 0.00% | 0.02% |
| Argon | 0.03% | 0.01% | 0.60% |
| Water | 7.14% | 2.30% | 0.83% |
| Ethylene Oxide | 32.14% | 10.33% | 3.72% |

The discharge gas stream (11) is directed to a countercurrent heat exchanger (12) where the sensible heat bulk of the sensible heat between the incoming hot gas (11) is exchange with the outgoing cold stream (27) thus reducing the thermal load on condenser 1 (14). Condenser 1 (14) may be cooled using either process cooling water or a higher temperature (greater than −40 degrees) refrigeration system using the primary refrigerant or a secondary refrigerant. The mix liquid-vapor stream (15) is feed into a liquid-gas separator (17) were the liquid ethylene oxide (18) is discharged to Ethylene oxide feed storage either by direct pressure or a liquid pump depending on the normal operating pressure of both systems. The vapor phase (19) is directed to a second condenser (20) which is operating at a lower temperature (−35 to −110 degrees). Additional ethylene oxide is condensed out. The liquid -vapor stream (21) is fed into a second liquid-vapor separator (22). The liquid stream (23) is fed into liquid-vapor separator (17) to be combined to produce liquid stream (18).

The vapor phase (24) is directed to flash valve (26) which produces a super-cooled gas stream (27) which is then fed into Heat exchange (12). The reheated gas stream (28) containing any unrecovered ethylene oxide is the mix with the dryer purge gas (38a) to form a mix stream (39) which is fed into an ethylene oxide mitigation system (40) prior to its release via stream (41) to the atmosphere or can be mixed with air stream (35) prior to the heater (36). The US EPA has regulated that 99% of the discharged ethylene oxide from the sterilization/fumigation chamber(s) has to be mitigated.

For regeneration of the Dryers (4a, 4b), air (30) is filtered by filter (31) then compressed by compressor (32). The compressed air stream (33) is directed to a flow control valve (34) which regulates the flow to the appropriate rate (35) before being directed to heater (36). The air temperature is increased to between 250 and 400 degrees Fahrenheit, this hot air stream (37a,37b) is directed via three way valves to flush the moisture laden dryer's (either 4a or 4b) molecular sieve of moisture. Once the desired bed temperature is achieved the flush with hot air is terminated and the bed is purged with the cool non-condensable gas (28). The purge discharge stream (38a, 38b) is directed to the ethylene oxide mitigation system.

We claim:

1. A method for recovering and recycling ethylene oxide from the vent feed gas stream of a sterilization/fumigation chamber where sterilization or fumigation process gas containing ethylene oxide, comprising:
a. a dryer for removing moisture contained in said vent feed gas stream;
b. a pressure booster where said vent feed gas stream pressure is increased to the desired operating pressure based on the required condensing temperature for ethylene oxide to achieve the desired recovery efficiency determined by the gas composition of said vent feed gas stream;
c. a heat exchanger to change the temperature of said vent feed gas stream to the desired operating condition to condense the ethylene oxide;
d. a container separating said gas stream condensable gases from the non-condensable portion of the said vent process gas stream;
e. a means of venting the non-condensable portion of said vent feed gas stream from the system; and
f. a means for recycling and storing for reuse the condensed ethylene oxide.
